(19) United States
(12) Patent Application Publication
(10) Pub. No.: US 2007/0253915 A1
(43) Pub. Date: Nov. 1, 2007

(54) METHODS FOR COATING DENTAL DEVICES WITH DRY-TO-THE-TOUCH SALIVA SOLUBLE FLAVORS

(75) Inventors: Dale G. Brown, Wharton, TX (US); Ira D. Hill, Austin, TX (US)

Correspondence Address: BANNER & WITCOFF, LTD., 28 STATE STREET, 28th FLOOR, BOSTON, MA, 02109-9601, US

(73) Assignee: WHITEHILL ORAL TECHNOLOGIES, INC., Stafford, TX (US)

(21) Appl. No.: 11/737,824

(22) Filed: Apr. 20, 2007

Related U.S. Application Data
(60) Provisional application No. 60/745,704, filed on Apr. 26, 2006.

(51) Int. Cl. A61K 8/89; A61K 9/68

(52) U.S. Cl. 424/48; 424/49; 427/2.14

(57) ABSTRACT

Methods for imparting a dry-to-the-touch, saliva soluble, coating containing high levels of flavors and active ingredients to disposable, one-handed, dental devices comprising imparting tumbling motion to said devices and simultaneously coating said tumbling devices with multiple coatings of the dry-to-the-touch-coatings, wherein said multiple coatings comprise from between about 0.25 and about 6% by weight of said multicoated devices.

### PRIORITY CLAIM

This application claims priority from U.S. Provisional Patent Application Ser. 60/745,704, filed Apr. 26, 2006, the disclosure of which is hereby incorporated herein by reference.

### FIELD OF INVENTION

The field of the present invention relates to methods for coating interproximal disposable, one-handed dental devices with dry-to-the-touch, saliva soluble flavors. These disposable, one-handed dental devices include a variety of medical devices suitable for cleaning spaces between teeth and below the gumline, including dental flossers, dental picks, dental massagers, proxy brushes, and the like. All of these biofilm therapy, disposable, one-handed dental devices can be treated with saliva soluble emulsions containing dry-to-the-touch saliva soluble flavors.

These coatings contain: (a) saliva-soluble flavor/film forming agent mixtures, or (b) saliva-soluble flavor/active ingredient/film forming agent mixtures, or (c) complex, saliva-soluble mixtures containing any or all of the above plus surfactants and/or emulsions, all of which are dry-to-the-touch according to the teachings of this invention. These coated devices are suitable for physically controlling, disrupting and removing biofilms, while delivering high levels of flavor and/or efficacious levels of active ingredients.

Specifically, the present invention relates to methods for improving disposable, one-handed dental devices with dry-to-the-touch, saliva soluble flavors, including:

(1) dental flossers as described in U.S. patents:

| | | | | |
|---|---|---|---|---|
| 147,987; | 175,795; | 413,001; | 2,217,917; | 2,059,287; |
| 2,784,722; | 1,815,408; | 2,187,899; | 3,974,842; | 3,926,201; |
| 2,187,899; | 2,443,415; | 4,615,349; | 3,974,842; | 4,006,750; |
| 4,016,892; | 5,086,792; | 5,538,023; | 5,692,531; | 3,858,594; |
| 2,180,522; | 2,187,899; | 2,443,415; | 3,926,201; | 4,615,349; |
| 1,415,762; | 2,702,555; | 2,811,162; | 3,693,594; | 4,192,330; |
| 4,522,216; | 4,807,651; | 5,113,885; | 5,123,432; | 5,538,023; |
| 5,503,168; | 5,483,982; | 5,388,600; | 5,287,865; | 5,246,021; |
| 5,738,125; | 5,704,379; | 5,904,155; | 5,829,458; | 6,065,479; |

6,227,210; and U.S. Patent Publication No. 2005/0205107 A1.

Additional examples of such devices are described in copending application Ser. No. 11/349,042, filed Feb. 7, 2006.

(2) dental picks as described in U.S. patents:

| | | | | |
|---|---|---|---|---|
| 4,651,760; | 4,805,646; | 5,234,009; | 1,527,028; | 2,008,206; |
| 3,101,172; | 3,910,293; | 4,135,528; | 4,271,854; | 4,314,574; |
| 4,403,625; | 4,570,653; | 4,577,649; | 4,942,034; | 2,667,443; |
| 2,748,781; | 2,772,205; | 2,896,639; | 3,838,702; | 3,897,795; |
| 4,029,113; | 4,175,326; | 4,462,136; | 4,510,127; | 4,627,975; |
| 4,616,667; | 5,234,009; | 1,527,028; | 2,008,206; | 3,101,172; |
| 3,910,293; | 4,135,528; | 4,271,854; | 4,314,574; | 4,403,625; |
| 4,570,653; | 4,577,649; | 4,942,034; | 2,667,443; | 2,748,781; |
| 2,772,205; | 2,896,639; | 3,838,702; | 3,897,795; | 4,029,113; |

| | | |
|---|---|---|
| 4,175,326; | 4,462,136; | 4,510,127; and 4,627,975. |

Additional examples of suitable other one-handed devices are described in co-pending patent application Ser. No. 11/349,042, filed Feb. 7, 2006.

### BACKGROUND OF THE INVENTION

Disposable, one-handed dental devices, including dental flossers and dental picks, are often preferred over traditional dental floss, because they require only one hand to insert into interproximal areas. Additionally, these convenient, portable, one-handed, disposable dental devices can be carried in pocket or purse, and/or stored in desk drawers, glove compartments, etc., and therefore are accessible for use throughout the day, after meals, snacks, coffee breaks, etc.

Because of the injection molding process used to manufacture dental flossers, they cannot be coated with substantial quantities of saliva soluble substances that can be released during flossing into the mouth. See U.S. patents on compression coating dental flosses and tapes, including:

| | | | | |
|---|---|---|---|---|
| 4,911,927; | 5,057,308; | 5,057,309; | 5,057,310; | 5,098,711; |
| 5,165,913; | 5,538,667; | 5,665,374; | 5,711,935; | 6,545,077; |
| 6,575,176; | 6,591,844; | 6,604,534; | 6,609,527; | 6,907,889; |

| | |
|---|---|
| 6,916,880; | 7,017,591; and 7,025,986 |

Specifically, the floss or dental tape substrate in flossers cannot be similarly coated prior to injection molding due to the high temperatures encountered in the injection molding process used to capture the floss or tape in flossers. These high temperatures will not accommodate coated floss substrates. Accordingly, disposable dental flossers generally are unflavored and deliver very little, if any, coating substances into interproximal areas during flossing.

Heretofore, most disposable flossers have had limited opportunity to introduce flavors, mouth feel agents, tingling substances, etc., into the oral cavity during use. Certain flossers have been provided with post-added zones of flavor, etc., such as described in U.S. Patent Application Publication 20060042650. To date, no disposable, one-handed, dental devices have been able to deliver tingling sensation, and/or mouthfeel to the oral cavity during use, such as described in copending patent application Ser. No. 11/349,042 filed on Feb. 7, 2006. Attempts to post-coat dental flossers and dental picks via dipping, spraying, etc., have generally been unsuccessful. Most post-coated devices have been unsightly and consumer unfriendly, indicating substantial flaking of the coating prior to use.

It is generally agreed that dental flossers, dental picks and other one-handed, disposable dental devices, including proxy brushes, could be improved substantially if they were coated with dry-to-the-touch, saliva soluble flavors.

### OBJECTS OF THE INVENTION

An object of the present invention is to coat disposable, one-handed dental devices with dry-to-the-touch, saliva soluble flavors.

A further object of the invention is to coat disposable, one-handed dental devices with multiple coatings of dry-to-the-touch, saliva soluble flavors, wherein the coated devices are consumer friendly.

Still another object of the invention is to adapt commercial confectionery equipment and processes to coating disposable, one-handed dental devices with multiple coatings of dry-to-the-touch, saliva soluble flavors.

Yet another object of the invention is to coat disposable, one-handed dental devices with multiple coatings of dry-to-the-touch, saliva soluble flavors/MICRODENT® emulsions.

Still another object of the invention is to coat disposable, one-handed dental devices with multiple coatings of saliva soluble flavor/film agent mixtures.

A further object of the invention is to coat disposable, one-handed dental devices with multiple coatings of saliva soluble flavor/active ingredient/film forming mixtures.

These and other objects of the invention are achievable and understandable by one skilled in the art after reviewing the specification and examples and claims set out below.

### SUMMARY OF THE INVENTION

The present invention is directed to a method for imparting a high flavor and mouth feel impact to disposable, one-handed, dental devices, comprising: imparting tumbling motion to said devices by means of a rotating drum-type means and simultaneously periodically introducing into the drum an emulsion, such as MICRODENT®, accompanied by controlled heat and air flow; thereby coating the devices in their entirety with a dry-to-the-touch, saliva soluble flavor coating, followed by drying each coating with heat and air prior to applying a subsequent coating and drying with heat and air. Said multiple coatings comprise from between about 0.25% and about 6% by weight of said multi-coated, disposable, one-handed dental device.

### Definitions

For purposes of describing the present invention, the following terms, as used throughout this specification, are defined as set out below:

“Disposable one-handed dental devices of the invention” are defined as medical devices treated with a dry-to-the-touch, saliva soluble flavor, or having a saliva soluble coating containing a dry-to-the-touch, saliva soluble flavor, which devices are suitable for physically controlling, disrupting and removing biofilms, while imparting a high flavor and mouth feel impact. Disposable, one-handed dental devices of the invention include: dental flossers, proxy brushes, dental stimulators, dental massagers, dental picks, etc., including:

1. Disposable, one-handed dental flossers such as described below:
For more than one hundred years there have been attempts to improve and modernize the dental flossing technique by development of holders onto which floss is detachably or permanently secured, and a few hundred patents have already been granted on various flossing devices. These go back to the Shurtleft U.S. Pat. No. 147,987 of 1874, and Wallace U.S. Pat. No. 175,795 of 1876. In general, the dental flossers of the prior art have fallen into two basic categories, namely the reusable or permanent dental flossers wherein the customer threads the device with floss and then throws away the floss after use, but retains the flosser; and the disposable dental flossers having a structure to which the floss is permanently attached, the entire device being thrown away after one or more uses and replaced by another similar device.
In general, the dental flossers of the permanent type are, as would naturally be expected, of considerably more complex construction and made of more expensive materials. Examples of such devices are those shown in the patents to Munroe U.S. Pat. No. 2,217,917; Storm U.S. Pat. No. 2,059,287; Chamberlin et al. U.S. Pat. No. 2,784,722; and Jordan U.S. Pat. No. 1,815,408.
Disposable, one-handed flossers, such as contemplated by the present invention, have to be made inexpensively and the floss has to be permanently attached to the flosser in some way. Examples of patents disclosing disposable flossers are the Chodorow U.S. Pat. No. 3,974,842; Katz U.S. Pat. No. 3,926,201; and Henne U.S. Pat. No. 2,187,899. Buscarino U.S. Pat. No. 2,443,415 shows both disposable and permanent flossers. A major problem with disposable flossers of the prior art is the inadequate means of attachment of the floss, because in all cases the prior art requires either complex and expensive, or insufficient means of attachment. Thus, the Chodorow and Katz patents require that the flosser be molded about the floss, which is extremely expensive. Knotting has also been suggested, but this also is expensive and in some cases inadequate. Henne U.S. Pat. No. 2,187,899 suggests various types of mechanical attachment, but none of these provide adequate anchoring; often when floss is forced between two adjacent teeth and the spacing is small, tremendous tensile force is applied and mechanical attachments of this type are not adequate, the floss ends merely pulling from their mechanical anchoring means.
With regard to the molding of the flosser about the ends of the floss, such as shown in the Chodorow and Katz patents, this produces not only a very expensive product, but also limits the types of floss which can be used. For example, medicated and flavored flosses, wherein the medicaments and flavorings are heat sensitive or volatile, cannot be used in the hot molding process because the heat of the operation and the molten plastic will degrade and/or drive off the medicament and/or flavoring material. Further, the medicaments and flavorings negatively impact the strength of the hot molded plastic as well as the polymer's ability to firmly affix the floss into the flosser device. See U.S. Patent Publication No. 2005/0205107 A1 and also the following U.S. patents:

| | | | | |
|---|---|---|---|---|
| 4,615,349, | 3,974,842; | 4,006,750; | 4,016,892; | 5,086,792; |
| 5,538,023; | 5,692,531; | 3,858,594; | 2,180,522; | 2,187,899; |
| 2,443,415; | 3,926,201; | 4,615,349; | 413,001; | 1,415,762; |
| 2,702,555; | 2,811,162; | 3,693,594; | 4,192,330; | 4,522,216; |
| 4,807,651; | 5,113,885; | 5,123,432; | 147,987; | 175,795; |
| 413,001; | 2,217,917; | 2,059,287; | 2,784,722; | 1,815,408; |
| 2,187,899; | 3,926,201; | 2,187,899; | 2,443,415; | 3,974,842; |
| 5,538,023; | 5,503,168; | 5,483,982; | 5,388,600; | 5,287,865; |
| 5,246,021; | 5,738,125; | 5,704,379; | 5,904,155; | 5,829,458; |

| | |
|---|---|
| 6,065,479; | 6,227,210; and U.S. Publication No. 2005/0205107 A1. |

Additional examples of such devices are described in copending application Ser. No. 11/349,042, filed Feb. 7, 2006.
All of the foregoing dental flosser references are incorporated herein by reference.

2. Dental picks of the invention such as described below:
In U.S. Pat. No. 4,651,760, a toothpick made from plastic is disclosed which has a pointed end supported by a mid portion having longitudinal notches which allows the toothpick to be compressed in a transverse direction and also has transverse flaps which may strike the tooth surfaces. A projecting plate is used as a handle. The flaps also insure that the toothpick attains considerable rigidity to keep its shape even though it may be subject to twisting.
In U.S. Pat. No. 4,805,646, a toothpick is disclosed which has a tapered triangular point which extends from a flexing joint which allows the point to assume various different angles. However, the tip itself if fairly rigid.
U.S. Pat. No. 4,616,667 discloses another toothpick design which has two ends which are pointed, having a shaft, a preferably substantially rectangular transverse cross-section and a longitudinally grooved cleaning tip which permits deformation of the tip in such a manner and direction to reach in between the narrow gaps of teeth while not sacrificing the longitudinal rigidity of the cleaning tip. Consequently, the area adjacent the tip may be bent but the tip itself remains rigid. See also U.S. patent Nos.:

| | | | | |
|---|---|---|---|---|
| 5,234,009; | 1,527,028; | 2,008,206; | 3,101,172; | 3,910,293; |
| 4,135,528; | 4,271,854; | 4,314,574; | 4,403,625; | 4,570,653; |
| 4,577,649; | 4,942,034; | 2,667,443; | 2,748,781; | 2,772,205; |
| 2,896,639; | 3,838,702; | 3,897,795; | 4,029,113; | 4,175,326; |
| 4,462,136; | 4,510,127; | 4,627,975; | 4,651,760; | 4,805,646; |
| 5,234,009; | 1,527,028; | 2,008,206; | 3,101,172; | 3,910,293; |
| 4,135,528; | 4,271,854; | 4,314,574; | 4,403,625; | 4,570,653; |
| 4,577,649; | 4,942,034; | 2,667,443; | 2,748,781; | 2,772,205; |
| 2,896,639; | 3,838,702; | 3,897,795; | 4,029,113; | 4,175,326; |
| 4,462,136; | 4,510,127; | 4,627,975; | 4,616,667; | 4,135,528; |

| | | |
|---|---|---|
| 4,271,854; | 4,314,574; | 4,403,625; and 4,570,653. |

### See Also the discussion of wooden toothpicks by Diamond Brands at their website, www.diamondbrands.com.

Additional examples of suitable other one-handed devices are described in co-pending patent application Ser. No. 11/349,042, filed Feb. 7, 2006.
All of the foregoing dental pick references are incorporated herein by reference.

“Dry-to-the-touch” coatings, suitable for coating the disposable, one-handed dental devices of the invention are defined as those compositions which, in spite of high levels of flavor oils, surfactants, waxy emulsions and active ingredients are, by reason of their formulation with dry-film forming agents surprisingly rendered dry-to-the-touch without reducing their saliva-soluble properties or limiting their ability to deliver consumer preferred properties when used in the oral cavity.

“Flavor reservoir” suitable for use with the various disposable, one-handed devices of the present invention is defined to include substances that adsorb (release) these volatile ingredients into surfactant and/or film forming ingredient containing coatings on the disposable, one-handed devices of the present invention. Examples of flavor reservoirs include adsorbent paper, adsorbent polymers such as polyvinylacetate (Elvax, supplied by DuPont) treated with volatile flavorants, and alcoholic solutions containing flavor oils.

“Wax emulsions” suitable for coating the disposable, one-handed dental devices of the invention are defined to include: (a) various water-soluble waxes such as PEG, alone or with a flavor solubilizing surfactant, (b) Lipowax (mixtures of fatty acid PEG surfactant, a long chain alcohol), and (c) emulsifying waxes.

“Saliva soluble coatings” suitable for the various disposable, one-handed, dental devices of the invention are defined as those saliva soluble substances coat disposable dental devices for purposes of: lubrication and ease of device insertion interproximally and for delivering interproximally, cleaners, therapeutic antimicrobials, flavors, and other additives. These saliva soluble coatings generally comprise from between about 0.25% to about 6% by weight of the disposable, one-handed dental device.

Preferred saliva soluble coatings which can contain dry-to-the-touch, saliva soluble flavors include:
(a) those emulsion coatings described in the following U.S. Pat. Nos. 4,950,479; 5,032,387; 5,538,667; 5,561,959; and 5,665,374, which are hereby incorporated by reference,
(b) various dental device coatings, such as described in U.S. Pat. Nos. 5,908,039; 6,080,495; 4,029,113; 2,667,443; 3,943,949; 6,026,829; 5,967,155 and 5,967,153, which are hereby incorporated by reference,
(c) those substantive saliva soluble coatings described and claimed in U.S. Pat. Nos. 6,907,889; 6,609,527; 6,916,880 and 6,545,077, which are hereby incorporated by reference, and
(d) those coatings described in copending applications: U.S. Ser. No. 11/096,606, filed Apr. 1, 2005, entitled: “Coated Monofilament Tape Bobbins and Methods for Winding”; U.S. Ser. No. 11/149,597, filed Jun. 10, 2005, entitled: “Non-Crystalline Saliva-Soluble Coatings for Elastomeric Monofilament Dental Tapes”; and U.S. Ser. No. 11/196,827, filed Aug. 3, 2005, entitled: “Biofilm Therapy Interproximal Devices.”

All of the foregoing saliva soluble coatings can contain biofilm-responsive levels of one or more substances suitable for controlling and disrupting biofilms and dry-to-the-touch, saliva soluble flavors.

As used herein, “coating” is generally defined as the process of introducing dry-to-the-touch, saliva soluble flavors, as well as other oral care substances onto the surfaces of various disposable, one-handed dental devices.

As used herein, the terms “MICRODENT®” and “ULTRAMULSION®” refer emulsions of polydimethylsiloxane at various molecular weights in various poloxamer surfactants as described and claimed in U.S. Pat. Nos. 4,911,927; 4,950,479; 5,032,387; 5,098,711; 5,165,913; 5,538,667; 5,645,841; 5,651,959 and 5,665,374. These mouth conditioners are preferably included in crystal-free emulsion coatings of various disposable, one-handed dental devices of the invention.

As used herein, the phrase “saliva-soluble, crystal-free coatings” refers to those saliva soluble emulsions that indicate substantial flake resistance, yet release from various disposable, one-handed dental devices of the present invention during use when exposed to saliva in the oral cavity. These coatings can include SOFT ABRASIVES® that are dispersed and not solubilized in said coatings. These SOFT ABRASIVES® remain insoluble when delivered between teeth and below the gum line during use. Additionally, saliva-soluble, crystal-free coatings preferably contain surfactants, mouth conditioners, chemotherapeutic ingredients and flavors that are released from the devices into the oral cavity, along with dry-to-the-touch, saliva soluble flavors. For example, see U.S. Pat. Nos. 6,609,527 and 6,575,176.

As used herein, the term “crystal-free” refers to a smooth surface on the device as distinguished from rough surfaces typical of crystalline coatings when observed through a 30× stereo zoom microscope. See U.S. Pat. No. 6,609,527. Generally, crystal-free coatings containing dry-to-the-touch, saliva soluble flavors indicate minimum flaking. Examples of suitable crystal-free coating formulations for various disposable, one-handed dental devices of the invention are detailed in the Examples below.

As used herein, the term “biofilm therapy disposable, one-handed dental devices” are defined as dental devices coated with dry-to-the-touch, saliva soluble flavors and emulsions such as MICRODENT®/ULTRAMULSION® that control, disrupt and physically remove biofilms, while imparting high flavor and mouth feel impact to the oral cavity. In a preferred embodiment of the invention, the coated biofilm therapy disposable, one-handed dental devices of the invention include a SOFT ABRASIVES® overcoating that is also released, along with the dry-to-the-touch, saliva soluble flavors, during use to work with the substrate to help physically remove biofilms. Working these disposable, one-handed dental picks, interproximally, massages interproximal soft tissues thereby increasing blood flow. The simultaneous release of dry-to-the-touch, saliva soluble flavors or various saliva soluble coatings containing dry-to-the-touch, saliva soluble flavors, while working these picks interproximally imparts high flavor and mouth feel impact to the oral cavity.

As used herein, the term “additional adjuvants” refers to additional ingredients that can be added to the dry-to-the-touch, saliva soluble flavors to provide color, or sweetening effects, as desired. Examples of suitable sweetening agents include sorbitol, sodium cyclamate, saccharine, commercial materials such as Nutrasweet® brand of aspartame and xylitol. Citric acid or acetic acid is often utilized as a flavor modifier and is generally used in amounts of about 1.0 to about 20 percent by weight, preferably about 2.0 percent to about 15 percent by weight.

As used herein, the term “buffering ingredient” refers to substances that may also be added to the dry-to-the-touch, saliva soluble flavors of the invention in order to prevent natural degradation of the flavoring components or therapeutically active ingredients. Generally, the pH of these compositions is adjusted from about 3.5 to about 8, depending on the chemistry of the active ingredient most requiring protection. Buffering ingredients such as an alkali metal salt of a weak organic acid, for instance, sodium benzoate, sodium citrate, sodium phosphate, sodium bicarbonate or potassium tartrate is generally added in an amount of about 0.1 to about 1.0 percent by weight. Other buffering agents such as weak organic acids or salts of weak bases and strong acids such as boric acid, citric acid, ammonium chloride, etc., can also be used in similar concentrations.

As used herein, the term “stabilizers” refers to substances that are often added along with the dry-to-the-touch, saliva soluble flavorant for additional control, such as:
(a) sodium benzoate, sodium or potassium sorbate, methyl paraben, propylparaben and others approved for ingestion, and
(b) chemical oxidative control substances, such as ethylene-diaminetetraacetic acid, BHA, BHT, propyl gallate and similar substances approved for ingestion. Concentration levels of these stabilizers comply with industry and regulatory standards.

As used herein, the term “SOFT ABRASIVES®” defines saliva-soluble and saliva-insoluble abrasive substances added to the coated devices of the invention that are suitable for cooperating with the various disposable, one-handed dental devices of the present invention to remove, control and disrupt biofilm, tartar and stained pellicle from tooth surfaces. SOFT ABRASIVES® include: tetrasodium pyrophosphate, calcium carbonate, dicalcium phosphate, silica, glass beads, polyethylene and polypropylene particles, pumice, titanium oxide, alumina, quartz, aluminum silicate, etc., at various particle sizes suitable for use in oral care. See U.S. Pat. No. 6,575,176.

As used herein, the term “cleaners” refers to essentially all surfactants suitable for use in the oral cavity and suitable for coating various disposable, one-handed dental devices of the present invention.

As used herein, the phrase “chemotherapeutic ingredients” refers to those substances other than dry-to-the-touch, saliva soluble flavors suitable for addition to the coatings of the present invention that impart therapeutic effects to the oral cavity including antimicrobials; anti-tartar and anti-plaque substances; remineralizing, desensitizing, NSAID and antibiotic ingredients, and the like. Specific chemotherapeutic ingredients suitable for the present invention include: stannous fluoride, potassium nitrate, cetylpyridinium chloride (CPC), triclosan, metronidazole, chlorhexidine, aspirin and doxycycline.

As used herein, the phrase “crystal formation eliminating additives” is defined as those coating additives that reduce, control and/or eliminate crystal formation and enhance the substantivity of the dry-to-the-touch, saliva soluble flavor-containing coating to disposable, one-handed dental devices of the invention when added to these coatings at modest levels. These include certain aliphatic, long chain, fatty alcohols having from between about 10 and 30 carbon atoms and/or various liquid surfactants such as polyethylene glycol sorbitan dialiphatic esters.

Aliphatic, long chain, fatty alcohols are suitable for the crystal-free, dry-to-the-touch, saliva soluble flavor coatings of the present invention. These can be represented by the structural formula ROH, wherein R represents a long chain alkyl group having from 20 to 30 carbon atoms. Specific examples include:

| | | |
|---|---|---|
| 1-decanol | 1-heptadecanol | 1-pentacosanol |
| 1-undecanol | 1-octadecanol | 1-hexacosanol |
| 1-dodecanol | 1-nonadecanol | 1-heptacosanol |
| 1-tetradecanol | 1-decosanol | 1-octacosanol |
| 1-pentadecanol | 1-henticosanol | 1-nonacosanol |
| 1-hexadecanol | 1-tricosanol | 1-triacosanol |

1-tetracosanol, and mixtures thereof.

Naturally occurring mixtures with substantial quantities of these fatty alcohols, or isomers thereof, including those chemically derived from natural sources also constitute suitable sources of aliphatic, long chain fatty alcohols for the purpose of this invention.

The long chain fatty alcohols can be purchased commercially from Stepan, Procter & Gamble and Aldrich Chemical Co. and a variety of companies processing vegetable and animal derived fatty alcohols.

### Methods for Coating Disposable, One-Handed Dental Devices

Various post-coating operations such as dipping, soaking and spraying have been used to coat dental flossers and dental picks. Unfortunately, the resultant coating levels are difficult to control as indicated by the substantial coating build up that is generally encountered during drying. This results in unpleasant-appearing, consumer unfriendly, coated devices that “turn-off” most consumers who try them.

Surprisingly, it has been found that various confectionery coating processes can be modified and controlled, when used with dry-to-the-touch, saliva soluble flavors for coating disposable, one-handed dental devices including dental picks, dental flossers and proxy brushes. The resultant multi-coatings of various dry-to-the-touch, saliva soluble flavor/surfactant mixtures impart high impact hedonics to these devices. These multi-coated dry-to-the-touch, saliva soluble flavor/surfactant dental devices are: not sticky to handle, pleasant to look at, easy to use, and most importantly deliver high impact flavor and mouth feel sensation to the oral cavity, along with prolonged, smooth mouthfeel that lasts for an extended period. Depending on the flavors used, these high-impact coatings can be made stable for prolonged periods and, accordingly, are commercially feasible for use on disposable, one-handed dental devices.

The primary mechanical requirement of any selected process and apparatus is the ability to suspend the dental devices in the air, or otherwise provide movement of the devices during the coating/drying operations which prevent the accumulation of the coating agents at the points of contact between the dental device and the apparatus which results in the “pooling” of the coating agents while they are still liquid and then drying in the “pooled” state. It will be obvious to one skilled in the art that allowing a manufacturing opportunity for droplets or areas of high concentration to occur at various places on the dental device during drying would be counterproductive to the intent of the invention.

There are several established confectionery coating processes and apparatus, which can be adapted by one skilled in the art to coat disposable, one-handed dental devices with dry-to-the-touch, saliva soluble flavors/surfactants/emulsions. These include:

(a) pan coating: both batch and continuous, as described in U.S. Pat. Nos. 5,010,838; 4,334,493; 3,911,860; 4,245,380; 3,448,718; 3,063,843; and 2,726,959. Additional pan coating references include:

For example, U.S. Pat. No. 4,334,493, to Okawara shows a rotary drum type apparatus for applying a coating to devices which includes a rotary drum supported by a frame for receiving a body of devices to be coated by spraying with a solvent. The drum which can be inclined through about 10-20″ in such a manner that its front surface is turned upwardly, includes a device which supplies a coating material into the interior of the drum and an inlet tube and an outlet tube to provide a supply of drying gas such as air to the interior. The support frame cooperates with an outer periphery of the drum to define an air suction duct disposed on the front side of the support frame and an air exhaust duct disposed on the rear side of the support frame so that a smooth flow of hot blast through the drum can be obtained.

Similarly, in U.S. Pat. No. 4,245,580 to Okawara, a device for coating granular solids is disclosed which has a double-cone rotary drum perforated so as to permit flow of air or gas into and out of the rotary drum, and which is tiltable about the axis at right angles to the axis of rotation of the rotary drum so that in the case of the discharge of product solids, the opening of the rotary drum may be directed downwardly. Axial annular insulating covers are providing which insure the effective thermal insulation of the rotary drum when the hot air or gas is blown thereinto. However, each of the Okawara disclosures discussed above relate to coating drums for batch operation since each body of cores to be coated must be fed into and discharged from a single opening. Similar operation and apparatus are shown in U.S. Pat. Nos. 3,448,718; 3,063,843; and 2,726,959.

U.S. Pat. No. 3,911,860 to Nohynek discloses a coating drum for continuous coating of dragees with a coating material and for subsequent application and glossing of a protective skin over the coating by use of a co-axially connected after-treatment drum. In particular, both drums are fixed in a scaling to each other and may be driven at different speeds by means of a variable speed transmission rotationally interconnected. The dragee drum shown by Nohynek is a double frustum, while the after-treatment drum is cylindrical. Both drums are equipped with a conveying baffles in order to push the product through from the entrance to the exit.

(b) fluidized bed/wurster machine coating: as described in U.S. Pat. Nos. 6,911,087; 3,196,827; 3,110,626; 4,330,502; 4,535,006; 5,236,503; 6,579,365.

In most of these fluidized bed techniques, the container acts as a processing zone in which the devices are processed, dried and/or treated. In order to optimize the processing of the devices, a certain spatial extent of the processing zones and/or of the extension of the fluidized bed is required, particularly with regard to the “flight altitude” of the devices. An analogous rule applies to fluidized bed granulation. One means of controlling the fluidized bed is by varying the gas flow into the processing zone. As is well known, this can be accomplished by manually changing the air volume, e.g., by means of an air slide, until the desired height of the fluidized bed has been achieved. The modification of the setting, as well as the monitoring of the fluidized bed zone, usually takes place visually by an operator. However, in order to maintain a constant altitude o the fluid bed zone, a continuous control is necessary, since, under certain circumstances during the treatment process, modifications of the material to be processed may occur that necessitate corresponding modifications of the “flight altitude” of the devices. It is also necessary to make different adjustments in situations where the material to be processed is different. Consequently, for good results during processing, expensive and cumbersome monitoring and manual setting by an operator are necessary. However, even careful monitoring by an operator cannot insure that an optimal setting is maintained continuously throughout the processing period. Optical illusions may affect the operator during visual control which can result in processing the devices in an undesirable manner. Additional fluidized bed references include:

U.S. Pat. No. 3,110,626 to G. L. Larson et al. discloses an apparatus whereby coating discrete solids suspended in a moving air stream is carried out within the interior region of a velocity concentration control element mounted in the base region of a funnel-shaped coating chamber. However such apparatus does not include any means for shielding the base of the spray pattern with an upwardly flowing column of air in order that the spray pattern may substantially develop before entrance thereinto of discrete solids to be coated.

U.S. Pat. No. 4,335,676 to Christian Debayeax et al. discloses a spouted bed granulating and/or coating apparatus wherein flow directing structure is provided to direct the gaseous flow stream in the upward direction for preventing contact and agglomeration of particles in the vicinity of the walls of the device. This patent fails to disclose structure by which the lower portion of the spray pattern is protected by an upwardly flowing column of air in order that the spray pattern may more fully develop before the entrance thereinto of particles to be coated.

U.S. Pat. No. 4,701,353 to Stanislaus M. P. Mutsers et al. discloses an apparatus whereby the liquid spray material is discharged out of a central channel as a vertically closed, conical film with a thrust exceeding the thrust of the gas streams for the purpose of causing the conical film to be nebulized to very fine droplets with the air of the surrounding gas stream. The resultant spray pattern is not protected about its initial base end by an upwardly moving column of air disposed thereabout.

U.S. Pat. No. 4,960,224 to Gustav A. Magg et al. discloses an atomizing nozzle constructed in a manner to eliminate the need to provide a metering pump or flow meter for each atomizing nozzle of an associated fluidized coating bed with the control of the flow through each atomizing nozzle being accomplished by varying the internal bone size of the flow control tubes. However, this patent fails to disclose structure for shielding the resultant spray pattern from immediate entrance thereinto of particles to be coated before the spray pattern is reasonably developed.

U.S. Pat. No. 4,858,552 to Werner Glatt et al. discloses an apparatus whereby a fluidized current carries particles, while still plastic, upwardly through a channel device for agglomerated material disposed at a distance above the perforated base causing the particles to impinge on the underside of a rotatable means provided for shaping the agglomerated material. The Glatt et al. apparatus does not disclose structure by which the particles to be coated are shielded against entry into the initially forming spray pattern.

U.S. Pat. No. 3,196,827 to D. E. Wurster et al. discloses a tubular partition defining an upbed therein into which an air and spray discharge pattern is directed and wherein a downbed of particles in near weightless suspension is disposed outwardly of the tubular partition, the spray nozzle being disposed below the bottom of the partition above the associated air distribution plate or screen. With this device, particles being coated are also free to immediately enter the lower beginning portion of the spray pattern.

(c) Dragee coating: as described in: Silesia Confiserie Manual #2 Special Handbook for Dragee and U.S. Pat. Nos. 5,171,589; 4,649,855; 3,831,262; 5,334,244; 3,095,326; 4,105,801; 4,753,790; 4,250,195; 3,554,767; 2,304,246; 2,460,698; 3,208,405; 3,635,735; 4,238,510 and British Patents 922,495 and 1,047,349.

The disposable, one-handed dental devices of the present invention can be coated with coatings containing dry-to-the-touch, saliva soluble flavors using various confectionery coating drums which introduce the mixture onto the surfaces of the dental devices while the devices are in motion within the coating drum. Two general types of machines can be adapted to the coating process of the present invention. The first tumbles dental devices within a horizontally rotatable drum while the dry-to-the-touch, saliva soluble flavor coatings are sprayed into the drum. The second uses a vertical flow of air to circulate the devices past a vertically disposed spray nozzle used to introduce the dry-to-the-touch, saliva soluble flavor coatings into the drum.

Three types of dragee coating processes that are adaptable to coating disposable, one-handed dental devices with dry-to-the-touch, saliva soluble flavors are detailed below:

1. Dragee Kettle:
For most applications the exact thickness of the coated layer is not critical and many different types of coating machines may be used to apply a crude, yet effective coating to the device. An older once popular type of coating machine is called a dragee kettle and examples of these machines are disclosed in U.S. Pat. Nos. 3,831,262 and 5,334,244. This machine includes a large drum-like vessel which is typically rotated about a horizontal axis. The vessel includes a coating chamber which is partially filled with devise to be coated so that as the vessel rotates, the devices roll and tumble along the inside wall of the coating chamber. During this tumbling motion, dry-to-the-touch, saliva soluble flavor coating emulsions in the form of aqueous suspensions of liquids are sprayed through nozzles and into contact with the rolling devices within the coating chamber. During the coating process, a current of temperature-controlled air circulates in the coating chamber of the dragee kettle, which helps evaporate the water of the coating emulsion so that the dry-to-the-touch, saliva soluble flavor/surfactant coating effectively dries and adheres to the devices.

One problem with the dragee kettle coating machine is that typically the devices are not the only surfaces coated within the coating chamber. Even when a carefully controlled spraying schedule is followed (such as spraying at very short intervals while the dragee kettle rotates), much of the sprayed coating material still ends up on the inside wall of the coating chamber, as well as throughout the evaporation/venting ducting. This over-spraying creates numerous contamination and cleaning problems, and further increases the cost of the coating since much of the coating material is lost during the coating process.

The above-described dragee kettle type coating machine is limited to coating dental devices which do not require much precision in the thickness of the multi-coated layer because the thickness of the coating of the devices will vary in the same batch. This process may be used to coat many different devices, as long as uniform coating distribution and thickness are not required.

2. Perforated Pan:
The next generation of device coating machines after the dragee kettle is called a perforated pan coating machine. This machine has improved the device coating process and is the most common type of dragee coating machine in use today. The perforated pan machine includes a rotatable perforated drum which rotates about a horizontal axis within a housing, and further includes a plurality of nozzles positioned within the drum. The nozzles create a spray of dry-to-the-touch, saliva soluble flavor within the drum so that any dental devices located within the drum will tumble about into and out of the spray pattern and, over a period time, will accumulate a coating throughout their surface. An important improvement of the perforated pan coating machine over the dragee kettle is that the perforated pan machine allows air directed through the housing (using appropriate ducting) to pass through the perforated drum and quickly reach the dental devices tumbling therein. The perforations of the drum effectively expose the tumbling devices to the current of air, resulting in more uniform distribution of drying air for each device. The drum further includes solid baffles which are used to enhance mixing of the device bed in an effort to improve the distribution of the dry-to-the-touch, saliva soluble flavor being sprayed onto the devices.

3. Fluidized Bed Coating Machines:
Another type of device coating apparatus is called a fluidized bed coating machine (also known as a Wurster machine, after inventor Dale Wurster). These have been discussed above. Several examples of the Wurster coating machine are disclosed in U.S. Pat. Nos. 3,196,827; 3,110,626; 3,880,116; 4,330,502; 4,535,006 and 5,236,503.
The Wurster coating machine is typically used to layer and coat granules or pellets of solid materials, including pharmaceutical drugs.
As described below, the Wurster machine generates an upward stream of air or other gases such as nitrogen to circulate a device through a vertical spray of dry-to-the-touch, saliva soluble flavor coating within a product container. As the dental device cycles through the spray, a minute amount of coating material is deposited on its surface. The number of cycles the dental device completes determines the thickness of the final coating layer.
One preferred embodiment, the present invention includes the use of drageeing kettles, where the axis of rotation is tilted to the horizontal. As a rule, such kettles have a pear-, tulip- or onion-shape in cross section. They are usually mounted on one side and have, on the side opposite to the mounting, a filling opening which, during operation, can be closed by a lid.
In such a kettle with an axis of rotation inclined to the horizontal, there is obtained a relatively complicated, three-dimensional movement, which, for example, is described in detail in an article by K. H. Bauer in “Pharmazeutische Industrie”, 39, 149-156/1977. Because of the fact that, in this case, the direction of action of the gravitational force differs from that of the centrifugal force or of the frictional force emanating from the walls of the container, in such a kettle the dry-to-the-touch, saliva soluble flavor coating in its deepest lying region is, roughly following the mantle line of the drageeing kettle, transported obliquely upwards as ascending material in the direction of the rotation of the kettle. It thereby obliquely reaches a zenith from which, as descending or running off material, it flows back counter to the rotational movement of the kettle. Because of the tilt of the kettle therefore, the descending material has a movement component towards the axis of the kettle so that, on average, it is closer to the axis of kettle rotation than the ascending material. As is to be seen from the above-cited article, due to this three-dimensional rolling movement of the dry-to-the-touch, salvia soluble flavor coating, there is achieved a better mixing up and thus a more uniform coating of the dental devices than in the case of the use of a kettle with a horizontal axis of rotation.
The dry-to-the-touch, saliva soluble flavor coating is periodically introduced into the coating drum, thereby applying successive coats to the dental devices which are being maintained in constant motion by the movement of the coating drums. Each coating application is followed up by a drying/tumbling interval during which substantially all of the moisture in the emulsion is expelled from the coated dental devices via the use of controlled air flow and the application of controlled heat. The duration the dry-to-the-touch, saliva soluble flavor coating is introduced into the coating drum usually ranges from between about 10 and about 120 seconds and preferably from between about 20 and about 75 seconds. The duration between sprays for drying the coating generally ranges from between about 2 and about 4 minutes. After this spray cycle, the dry-to-the-touch, saliva soluble coating on the dental devices loses substantially all of the moisture as the discrete dry-to-the-touch, saliva soluble flavor coating layer forms and dries. Care must be taken not to overdry or overheat the coated devices before a subsequent coating is added. Evidence of overheating or overdrying is the presence of flakes of coating material in the pan coating drum.

4. Vibrating Screen Coating Machines:
Another type of device coating apparatus is called a vibrating screen drying machine. In such an apparatus, the items to be coated and dried are conveyed through a drying section, providing a flow of either cool air or warm air, on a vibrating screen which “bounces” the parts into the air by the high amplitude vibration of the screen. The coating material can be applied by a variety of methods, two examples of which will suffice to illustrate the breadth of possibilities available to one skilled in the art. (1) the parts are immersed into the solution and transferred to a draining screen or screen conveyer for the removal of excess liquid before being transported to the vibrating screen (itself often in the form of a moving conveyer) for drying, or (2) spray devices are placed at strategic locations along the moving, vibrating screen to apply a series of “coats” between drying stages.
The total number of dry-to-the-touch, saliva soluble flavor coatings on the disposable, one-handed devices of the invention generally ranges from between about 1 and about 50 coatings and preferably from between about 4 and about 10 coatings.

All of the various confectionery coating apparatus and methods described above can be adapted by one skilled in the art to coat disposable, one-handed dental devices with dry-to-the-touch, saliva soluble flavors.

### PREFERRED EMBODIMENTS OF THE INVENTION

Preferably, the surfactant is included with a film forming polydimethylsiloxane, i.e., MICRODENT® or ULTRAMULSION®. As used herein, the terms “MICRODENT®” and “ULTRAMULSION®” refer emulsions of polydimethylsiloxane at various molecular weights in various poloxamer surfactants as described and claimed in U.S. Pat. Nos. 4,911,927; 4,950,479; 5,032,387; 5,098,711; 5,165,913; 5,538,667; 5,645,841; 5,651,959 and 5,665,374. These mouth conditioners are preferably included in crystal-free contact coatings of various disposable one-handed dental devices of the invention.

In a particularly preferred embodiment of the invention, the dry-to-the-touch, saliva soluble flavor coatings of the invention also contain a film-forming agent. Preferably, at least one film-forming agent is utilized in the preparation of the coating mixture. Representative film-forming agents include hydroxypropyl cellulose, methyl cellulose (i.e., methyl ether of cellulose), ethyl cellulose, hydroxypropyl methyl cellulose, hydroxymethyl cellulose, carboxymethyl cellulose, gelatin, mixtures thereof, and the like. Preferably, a branched chained film-forming agent such as hydroxypropyl cellulose, is utilized. Preferably, the hydroxypropyl cellulose has a Brookfield viscosity of not less than 145 cps for a 10% aqueous solution at 25° C. The coating emulsion can contain more than one film-forming agent and as such, for example, hydroxypropyl cellulose and methyl cellulose may be utilized. In such a combination the branched chained film-forming agent (e.g., hydroxypropyl cellulose) can be used in amounts of about 0.05 to about 1.5 wt. % with about 0.01 to about 0.5 wt. % being preferred, and the straight chained film-forming agent (e.g., methyl cellulose) can be used in amounts of about 0.5 about 1.0 wt. % with about 0.1 to about 0.5 wt. % being preferred.

Another particularly preferred embodiment of the invention utilizes the unique flavor adsorbing and retention properties of various dry-film forming agents and various surfactants, as described above. The modified cellulose film forming agent and the surfactants function as attractants for flavor molecules, to such an extent that they will adsorb and hold a high percentage of flavor molecules even from volatile sources after the film formers and surfactants have been coated onto the devices of the invention. This property of adsorbing and holding flavor molecules provides much of the consumer satisfaction associated with the present invention, as adsorbed flavor molecules are released upon the coatings dissolving in the saliva, thereby releasing the flavorings accessible to olfactory organs in the oral cavity.

This can be accomplished by coating devices of the invention with formulation minus the desired flavors and drying the coated devices by one of the mechanical procedures described above, followed by a final step of introducing volatile flavoring agents from the flavor reservoir. The coated devices can then be packed off either in bulk or consumer packaging material which is a sufficient barrier to flavor molecules to allow the flavors to be volatilized inside the container. This is easily accomplished by placing the desired quantity of volatile flavor oils into a flavor reservoir such as on a piece of adsorbent paper, or into a polymer such as polyvinylacetate (Elvax as supplied by Dupont) which adsorbs/desorbs at a high rate, into the package. Equilibrium is quickly established so that the bulk of the flavor moves from the flavor reservoir into the dry-film forming agents and/or surfactants present in the coated devices.

In another embodiment, to reduce the loss of volatile flavor molecules during the high temperature/high velocity air contact of the coatings during drying with the dragee method, the dental devices are coated to the desired thickness with a dry-film forming composition minus the volatile flavoring agents. As a last step, an alcoholic solution of the flavor oils is sprayed or otherwise introduced onto the moving dental devices in the dragee kettle for distribution across the surfaces. In the same manner that an alcoholic solution of perfume oils can be applied to the skin and dry off leaving almost all the perfume oils adsorbed into the skin, the alcoholic carrier of the flavor oils volatilizes off at very low temperatures which essentially eliminates the loss of volatile flavor oils. The thin film of flavor oils so deposited on the previously laid down device coating is almost instantly adsorbed due to the adsorption properties of the dry-film forming agents and/or surfactants. Surprisingly, any dis-uniformity in flavor oil across the surface of the disposable, coated, one-handed dental device is quickly made uniform by the inexorable principles of equilibrium as the oils move from points of higher concentration in the coating to those of lower concentration until equilibrium is reached.

It is self-evident in the forgoing preferred embodiments that non-volatile flavor components, such as sweeteners, must be added to the initial un-flavored coats as they cannot be transferred easily by equilibrium techniques.

Other substances can be added to the dry-to-the-mouth, saliva soluble flavor coatings of the invention including:
(a) A flavoring agent may be present in the emulsion in an amount within the range of from about 0.1 to about 10.0 wt. %, and preferably from about 0.5 to about 3.0 wt. %, of the emulsion. The flavoring agents may comprise essential oils, synthetic flavors, or mixtures thereof including, but not limited to, oils derived from plants and fruits such as citrus oils, fruit essences, peppermint oil, spearmint oil, clove oil, oil of wintergreen, anise and the like. Artificial flavoring components are also contemplated for use in coating emulsions of the present invention. Those skilled in the art will recognize that natural and artificial flavoring agents may be combined in any sensorially acceptable blend. All such flavor sand flavor blends are contemplated by the present invention.
(b) The colorant used may include dyes, pigments, lakes and natural colors. The colorant may be blended with melted wax, preferably carnauba wax, which melts at 80°-90° C., then cooled and ground to a find particle size that will pass at least 99% through a #100 mesh sieve (less than 150 microns). Other waxes that may be used include beeswax, candelilla wax, spermaceti wax, and mixtures of the foregoing. Another method of blending is to powder blend the colorant with the wax. In either method, the preferred ratios are 1-30% colorant and 70.99% wax, and more preferably 5-15% colorant and 85-95% wax. It is preferable to use powdered colorants that have a particle size that will pass at least 99.9% through a #325 mesh sieve (small than 45 microns) so that the blended color/wax will still pass at least 99% through a #100 mesh sieve, having a particle size of 150 microns or less.
(c) Artificial sweeteners such as the soluble saccharin salts, i.e., sodium or calcium saccharin salts, cyclamate salts, acesulfam-K, and the like, and the free acid form of saccharin may optionally be added to the dry-to-the-touch, saliva soluble flavor coatings of the invention. Dipeptide sweeteners such as L-aspartyl-L-phenylalanine methyl ester and materials described in U.S. Pat. No. 3,492,131, and the like may also be used. These sweeteners may be used in amounts of about 0.005 wt. % to about 0.5 wt. % based on the weight of the total coating emulsion, and preferably about 0.05 wt. % to about 0.25 wt. %. Usually the first coating emulsion can contain about 0.02 wt. % to about 0.06 wt. % and most preferably 0.05 wt. % of artificial sweetener. A second emulsion can usually contain about 0.05 wt. % to about 0.2 wt. %, based on the weight of the coating emulsion, with about 0.08 wt. % to about 0.15 wt. % being preferred of artificial sweetener.
(d) Flavoring in the coating emulsion will be present in an amount within the range of from about 0.5 to about 5% and preferably from about 1.25 to about 4% by weight of the emulsion. Such flavoring may comprise oils derived from plants, leaves, flowers, fruit, etc. Representative flavor oils of this type include citrus oils such as lemon oil, orange oil, lime oil, grapefruit oil, fruit essences such as apple essence, pear essence, peach essence, strawberry essence, apricot essence, raspberry essence, cherry essence, plum essence, pineapple essence, as well as the following essential oils: peppermint oil, spearmint oil, mixtures of peppermint oil and spearmint oil, clove oil, bay oil, anise oil, eucalyptus oil, thyme oil, cedar leaf oil, cinnamon oil, oil of nutmeg, oil of sage, oil of bitter almonds, cassia oil, and methylsalicylate (oil of wintergreen). Various synthetic flavors, such as mixed fruit, may also be incorporated in the dry-to-the-touch, saliva soluble flavor coatings of the invention with or without conventional preservatives.

In a particularly preferred embodiment of the invention, the polyalcohol, xylitol in a mixture with mono-, di- and triglycerides of the fatty acids of: palmitic, stearic and oleic acids, is included in the dry-to-the-touch, saliva soluble flavor coatings of the invention. Xylitol can be represented by the structured formula:

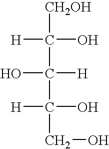

Xylitol is commercially available both in solid form and in the form of aqueous concentrated solutions. However, contrary to the saccharose shell, that of xylitol deteriorates rapidly with time. In particular, already after a few hours a xylitol shell cracks and its outer surface initially smooth becomes wrinkled; at the same time even the intimate constitution of the shell, initially sufficiently microcrystalline, changes into a course, rough structure fastidious to the palate and in chewing. A xylitol/dry-to-the-touch, saliva soluble flavor coating offers the peculiar advantage of being refreshing to the mouth (owing to an appreciable negative heat of solution); however, this advantage does not compensate the aforesaid drawbacks.

Those mixtures of glycerides are preferred which exhibit a strong prevalence of a determined glyceride. Mono- and diglycerides suitable for the purposes of this invention may present a melting point ranging from about 40° to about 70° C., keeping in mind that the melting point can be lowered (owing to formation of eutectics) by addition of a triglyceride having a convenient melting point. The preferred fatty substance is cocoa butter which, as is known, melts around 35° C.

The invention will be further understood by those skilled in the art upon reviewing the Examples set forth below.

### EXAMPLE 1

Fifteen grams of Klucel LF (hydroxypropylcellulose) is added slowly to 100 grams of water heated to 90 degrees centigrade. A solution of PEG 1450 (300 grams water with 10 grams PEG) was heated to 40 degrees centigrade. Spearmint flavor, 10 grams, was added with stirring along with 3 grams of sodium saccharin added to the PEG solution. The Klucel suspension was cooled to 50 degrees centigrade and the PEG solution added with stirring. The resulting solution is then sprayed in 5 aliquots with 4 minute intervals onto 1,000 plastic flossers contained in a rotating drum fitted with fins and a hot air tube delivering 40 degree air while tumbling at 21 RPM. The flossers were tumbled with hot air until the glistening effect of liquid coating was not evident after each aliquot. The coated flossers were very flavorful and had a coating of 14 mg per flosser.

### EXAMPLE 2

An aqueous suspension of hydroxypropylcellulose (100 grams water with 15 grams HPC) is heated to 90 degrees centigrade. A second container is heated with 300 grams water and 12 grams poloxamer 407 with heating to 40 degrees centigrade. Ten grams of flavor, vanillamint, is then added to the surfactant solution along with 4 grams of sodium saccharin. The aqueous HPC solution is cooled to 50 degrees and the surfactant solution is then added with stirring. After cooling to 30 degrees centigrade the combined solutions are sprayed in 4 aliquots with 5 minute drying intervals onto 10,000 round wooden toothpicks on a vibrating screen. Warm air at 40 degrees centigrade is directed up through the screen while the vibrating toothpicks are dried. The toothpicks have a coating of 2.4 mg each and show no marks or lines due to uneven coating.

What is claimed is:

1. A method for coating disposable, one-handed dental devices with dry-to-the-touch, saliva soluble coatings containing high levels of flavors and active ingredients, comprising:
a. introducing said devices into a moving coating drum that imparts tumbling motion to said dental devices,
b. introducing, periodically into said drum, a dry-to-the-touch, saliva soluble, coating mixture containing high levels of flavors and active ingredients,
c. coating said moving dental devices with coatings of said dry-to-the-touch, saliva soluble, coating mixtures,
d. removing substantially all the moisture between coatings, and
e. discharging said coated dental devices from said coating drum, at such time as said dry-to-the-touch coating comprises from between about 0.25 and about 6% by weight of said coated devices.

2. An apparatus useful for coating disposable, one-handed dental devices with multiple dry-to-the-touch saliva soluble coatings that includes: (a) a coating drum arrangement, which preferably rotates around an axis, keeping dental devices introduced into said drum in motion, (b) a means for periodically introducing a dry-to-the-touch, saliva soluble, coating mixture containing high levels of flavors and active ingredients into said drum at a controllable rate, and (c) controllable heat and air flow means for removing moisture from said dry-to-the-touch coated dental devices between coatings as they are tumbled in said coating drum.

3. A method for coating disposable, one-handed dental devices with dry-to-the-touch, saliva soluble, coating mixtures containing high levels of flavors and active ingredients comprising: introducing said devices into a coating vessel that imparts motion to said devices, periodically introducing said dry-to-the-touch, saliva soluble, coating mixtures containing high levels of flavors and active ingredients into said coating vessel, and removing moisture from said vessel between coating applications with the introduction of controllable heat and air flow.

4. A method according to claim 1, wherein said dry-to-the-touch, saliva soluble, coating mixtures containing high levels of flavors and active ingredients contains a surfactant selected from the group consisting of solid and liquid surfactants and mixtures thereof.

5. A method according to claim 1, wherein said emulsion contains a polydimethylsiloxane.

6. A method according to claim 1, wherein said dry-to-the-touch, saliva soluble, coating mixtures contains a film former.

7. Disposable, one-handed dental devices produced by adapting confectionery coating processes, including:
a. introducing said devices into a moving coating drum that imparts tumbling motion to said dental devices,
b. introducing, periodically into said drum, a dry-to-the-touch, saliva soluble, coating mixture containing high levels of flavors and active ingredients,
c. coating said moving dental devices with said coating mixtures,
d. removing substantially all the moisture between coatings, and
e. discharging said dry-to-the-touch coated dental devices from said coating drum, as such time as said coating comprises from between about 0.25 and about 6% by weight of said coated devices.

8. In confectionery coating technology, the improvement comprising introducing disposable, one-handed dental devices into a rotating confectionery coating drum and periodically introducing dry-to-the-touch, saliva soluble, coating mixtures containing high levels of flavors and active ingredients to coat said devices with multiple coatings of dry-to-the-touch, saliva soluble, uniform coating mixtures containing high levels of flavors and active ingredients.
